(19) United States
(12) Patent Application Publication
(10) Pub. No.: US 2010/0029652 A1
(43) Pub. Date: Feb. 4, 2010

(54) Combination Therapy For Treating Hepatitis C Infections

(75) Inventors: Mingjun Huang, Potomac, MD (US); Joanne Fabrycki, Southington, CT (US); Wengang Yang, Cheshire, CT (US); Xingtie Nie, West Haven, CT (US); Avinash Phadke, Branford, CT (US); Milind Deshpande, Madison, CT (US)

Correspondence Address: CANTOR COLBURN, LLP, 20 Church Street, 22nd Floor, Hartford, CT, 06103, US

(73) Assignee: ACHILLION PHARMACEUTICALS, INC., New Haven, CT (US)

(21) Appl. No.: 12/519,470

(22) PCT Filed: Dec. 17, 2007
(86) PCT No.: PCT/US07/25907

§ 371 Date: Jun. 16, 2009

(51) Int. Cl. A61K 31/5377; A61K 31/17; A61K 31/44; A61K 31/497; A61K 31/4965

(52) U.S. Cl. 514/237.2; 514/584; 514/355; 514/253.13; 514/255.06

(57) ABSTRACT

The invention provides a method of treating hepatitis C, by providing compound of Formula I (shown below) in combination with a second active agent to a patient infected with the hepatitis C virus. Also provided herein are pharmaceutical combinations comprising a compound of Formula I and a second active agent. The pharmaceutical combination may be a unit dosage form or a packaged pharmaceutical composition. Packaged pharmaceutical combinations include a compound of Formula I and a second active agent in a package with instructions for using the formulation to treat a hepatitis C infection.

### FIELD OF THE INVENTION

This invention provides a method of treating hepatitis C, by providing compound of Formula I (shown below) in combination with at least one additional active agent to a patient infected with the hepatitis C virus. Also provided herein are pharmaceutical combinations comprising a compound of Formula I and at least one additional active agent. The pharmaceutical combination may be a unit dosage form or a packaged pharmaceutical composition.

### BACKGROUND

An estimated 3% of the world's population is infected with the hepatitis C virus. Of those exposed to HCV, 80% become chronically infected, at least 30% develop cirrhosis of the liver and 1-4% develop hepatocellular carcinoma. Hepatitis C Virus (HCV) is one of the most prevalent causes of chronic liver disease in the United States, reportedly accounting for about 15 percent of acute viral hepatitis, 60 to 70 percent of chronic hepatitis, and up to 50 percent of cirrhosis, end-stage liver disease, and liver cancer. Chronic HCV infection is the most common cause of liver transplantation in the U.S., Australia, and most of Europe. Hepatitis C causes an estimated 10,000 to 12,000 deaths annually in the United States. While the acute phase of HCV infection is usually associated with mild symptoms, some evidence suggests that only about 15% to 20% of infected people will clear HCV.

HCV is an enveloped, single-stranded RNA virus that contains a positive-stranded genome of about 9.6 kb. HCV is classified as a member of the Hepacivirus genus of the family Flaviviridae. At least 4 strains of HCV, GT-1-GT-4, have been characterized.

The HCV lifecycle includes entry into host cells; translation of the HCV genome, polyprotein processing, and replicase complex assembly; RNA replication, and virion assembly and release. Translation of the HCV RNA genome yields a more than 3000 amino acid long polyprotein that is processed by at least two cellular and two viral proteases. The HCV polyprotein is:

NH2-C-E1-E2-p7-NS2-NS3-NS4A-NS4B-NS5A-NS5B-COOH.

The cellular signal peptidase and signal peptide peptidase have been reported to be responsible for cleavage of the N-terminal third of the polyprotein (C-E1-E2-p7) from the nonstructural proteins (NS2-NS3-NS4A-NS4B-NS5A-NS5B). The NS2-NS3 protease mediates a first cis cleavage at the NS2-NS3 site. The NS3-NS4A protease then mediates a second cis-cleavage at the NS3-NS4A junction. The NS3-NS4A complex then cleaves at three downstream sites to separate the remaining nonstructural proteins. Accurate processing of the polyprotein is asserted to be essential for forming an active HCV replicase complex.

Once the polyprotein has been cleaved, the replicase complex comprising at least the NS3-NS5B nonstructural proteins assembles. The replicase complex is cytoplasmic and membrane-associated. Major enzymatic activities in the replicase complex include serine protease activity and NTPase helicase activity in NS3, and RNA-dependent RNA polymerase activity of NS5B. In the RNA replication process, a complementary negative strand copy of the genomic RNA is produced. The negative strand copy is used as a template to synthesize additional positive strand genomic RNAs that may participate in translation, replication, packaging, or any combination thereof to produce progeny virus. Assembly of a functional replicase complex has been described as a component of the HCV replication mechanism. Provisional application 60/669,872 “Pharmaceutical Compositions and Methods of Inhibiting HCV Replication” filed Apr. 11, 2005, is hereby incorporated by reference in its entirety for its disclosure related to assembly of the replicase complex.

Current treatment of hepatitis C infection typically includes administration of an interferon, such as pegylated interferon (IFN), in combination with ribavirin. The success of current therapies as measured by sustained virologic response (SVR) depends on the strain of HCV with which the patient is infected and the patient's adherence to the treatment regimen. Only 50% of patients infected with HCV strain GT-1 exhibit a sustained virological response. Direct acting antiviral agents such as ACH-806, VX-950 and NM 283 (prodrug of NM 107) are in clinical development for treatment of chronic HCV. Due to lack of effective therapies for treatment for certain HCV strains and the high mutation rate of HCV, combinations of active agents with complimentary mechanisms of action may be needed to suppress emergence of viral resistance. The present invention fulfills this need and provides additional advantages, which are described herein.

### SUMMARY OF THE INVENTION

The invention provides a method of treating hepatitis C comprising providing a compound of Formula I or a pharmaceutically acceptable salt thereof, with at least one additional active agent to a patient infected with a hepatitis C virus, wherein Formula I is

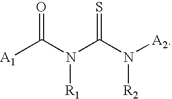

Within Formula I, the variables A₁, R₁, R₂, and A₂ carry the following definitions.

A₁ is an optionally substituted 3-pyridyl;

R₁ and R₂ are independently chosen from hydrogen and C₁-C₆alkyl; and A₂ is phenyl substituted at the para position with C₃-C₈alkyl or C₃-C₈alkoxy and optionally substituted with one or more substituent independently chosen from halogen, hydroxy, cyano, C₁-C₆alkyl, C₁-C₆alkoxy, mono- and di-(C₁-C₆alkyl)amino, C₂-C₆alkanoyl, C₁-C₂haloalkyl, C₁-C₂haloalkoxy, and phenyl.

The invention also provides pharmaceutical combinations comprising a compound of Formula I (as shown above) or a pharmaceutical acceptable salt thereof and at least one additional active agent. The compound of Formula I may be provided in a single dosage form, or may be formulated as separate dosage forms and provided together as separate dosage forms. For example the compound of Formula I and at least one additional active agent may be provided together in a container as a packaged pharmaceutical combination. Such packaged combinations may contain instructions for using the combination to treat a hepatitis C infection.

### BRIEF DESCRIPTION OF THE DRAWINGS

FIG. 1. Genome structures of HCV replicons. a) Prototype G418-selectable replicon 1377/NS3-3′1 and b) Bicistronic G418-selectable replicon I389luc-ubi-neo/NS3-3′/ET expressing a luciferase reporter and containing three adaptive mutations (E1202G, T12801, K1846T; arrows).

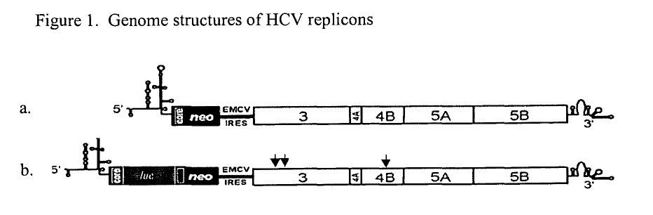

FIG. 2. Remaining colonies after 9-days treatment. The assay was conducted as described below. The numbers of colonies for untreated and single agent treated plates were estimated after dividing the plates into 8 equal portions and counting ever other portions (total 4 portions each).

### DETAILED DESCRIPTION OF THE INVENTION

### Terminology

The terms “a” and “an” do not denote a limitation of quantity, but rather denote the presence of at least one of the referenced items. The term “or” means “and/or”. The terms “comprising”, “having”, “including”, and “containing” are to be construed as open-ended terms (i.e., meaning “including, but not limited to”). Recitation of ranges of values are merely intended to serve as a shorthand method of referring individually to each separate value falling within the range, unless otherwise indicated herein, and each separate value is incorporated into the specification as if it were individually recited herein. The endpoints of all ranges are included within the range and independently combinable. All methods described herein can be performed in a suitable order unless otherwise indicated herein or otherwise clearly contradicted by context. The use of any and all examples, or exemplary language (e.g., “such as”), is intended merely to better illustrate the invention and does not pose a limitation on the scope of the invention unless otherwise claimed. No language in the specification should be construed as indicating any non-claimed element as essential to the practice of the invention as used herein. Unless defined otherwise, technical and scientific terms used herein have the same meaning as is commonly understood by one of skill in the art to which this invention belongs.

An “active agent” means a compound (including a compound of Formula I), element, or mixture that when administered to a patient, alone or in combination with another compound, element, or mixture, confers, directly or indirectly, a physiological effect on the patient. The indirect physiological effect may occur via a metabolite or other indirect mechanism. When the active agent is a compound, then salts, solvates (including hydrates) of the free compound or salt, crystalline forms, non-crystalline forms, and any polymorphs of the compound are included. Compounds may contain one or more asymmetric elements such as stereogenic centers, stereogenic axes and the like, e.g., asymmetric carbon atoms, so that the compounds can exist in different stereoisomeric forms. These compounds can be, for example, racemates or optically active forms. For compounds with two or more asymmetric elements, these compounds can additionally be mixtures of diastereomers. For compounds having asymmetric centers, all optical isomers in pure form and mixtures thereof are encompassed. In addition, compounds with carbon-carbon double bonds may occur in Z- and E-forms, with all isomeric forms of the compounds. In these situations, the single enantiomers, i.e., optically active forms can be obtained by asymmetric synthesis, synthesis from optically pure precursors, or by resolution of the racemates. Resolution of the racemates can also be accomplished, for example, by conventional methods such as crystallization in the presence of a resolving agent, or chromatography, using, for example a chiral HPLC column. All forms are contemplated herein regardless of the methods used to obtain them.

All forms (for example solvates, optical isomers, enantiomeric forms, polymorphs, free compound and salts) of an active agent may be employed either alone or in combination.

As used herein, “alkyl” includes both branched and straight chain saturated aliphatic hydrocarbon groups, having the specified number of carbon atoms, generally from 1 to about 12 carbon atoms. The term C₁-C₆alkyl as used herein indicates an alkyl group having from 1 to about 6 carbon atoms. When C₀-Cₙ alkyl is used herein in conjunction with another group, for example, arylC₀-C₄ alkyl, the indicated group, in this case aryl, is either directly bound by a single covalent bond (C₀), or attached by an alkyl chain having the specified number of carbon atoms, in this case from 1 to about 4 carbon atoms. Examples of alkyl include, but are not limited to, methyl, ethyl, n-propyl, isopropyl, n-butyl, 3-methylbutyl, t-butyl, n-pentyl, and sec-pentyl.

“Alkanoyl” indicates an alkyl group as defined above, attached through a keto (—(C═O)—) bridge. Alkanoyl groups have the indicated number of carbon atoms, with the carbon of the keto group being included in the numbered carbon atoms. For example a C₂alkanoyl group is an acetyl group having the formula CH₃(C═O)—.

The term “mono- and/or di-alkylamino” indicates secondary or tertiary alkyl amino groups, wherein the alkyl groups are as defined above and have the indicated number of carbon atoms. The point of attachment of the alkylamino group is on the nitrogen. The alkyl groups are independently chosen. Examples of mono- and di-alkylamino groups include ethylamino, dimethylamino, and methyl-propyl-amino. “Mono- and/or dialkylaminoalkyl” groups are mono- and/or di-alkylamino groups attached through an alkyl linker having the specified number of carbon atoms, for example a di-methylaminoethyl group. Tertiary amino substituents may by designated by nomenclature of the form N—R—N—R′, indicating that the groups R and R′ are both attached to a single nitrogen atom.

As used herein “haloalkyl” indicates both branched and straight-chain alkyl groups having the specified number of carbon atoms, substituted with 1 or more halogen atoms, generally up to the maximum allowable number of halogen atoms. Examples of haloalkyl include, but are not limited to, trifluoromethyl, difluoromethyl, 2-fluoroethyl, and penta-fluoroethyl.

“Haloalkoxy” indicates a haloalkyl group as defined above attached through an oxygen bridge (oxygen of an alchol radical).

“Halo” or “halogen” as used herein refers to fluoro, chloro, bromo, or iodo.

The phrase “optionally substituted” indicates that such groups may either be unsubstituted or substituted at one or more of any of the available positions, typically 1, 2, 3, or 4 positions, by one or more suitable groups such as those disclosed herein.

Suitable groups that may be present on a substituted position include, but are not limited to, e.g., halogen; cyano; hydroxyl; nitro; azido; alkanoyl (such as a C₂-C₆ alkanoyl group such as acyl or the like); carboxamido; alkyl groups (including cycloalkyl groups, having 1 to about 8 carbon atoms, or 1 to about 6 carbon atoms); alkenyl and alkynyl groups (including groups having one or more unsaturated linkages and from 2 to about 8, or 2 to about 6 carbon atoms); alkoxy groups having one or more oxygen linkages and from 1 to about 8, or from 1 to about 6 carbon atoms; aryloxy such as phenoxy; alkylthio groups including those having one or more thioether linkages and from 1 to about 8 carbon atoms, or from 1 to about 6 carbon atoms; alkylsulfinyl groups including those having one or more sulfinyl linkages and from 1 to about 8 carbon atoms, or from 1 to about 6 carbon atoms; alkylsulfonyl groups including those having one or more sulfonyl linkages and from 1 to about 8 carbon atoms, or from 1 to about 6 carbon atoms; aminoalkyl groups including groups having one or more N atoms and from 1 to about 8, or from 1 to about 6 carbon atoms; aryl having 6 or more carbons and one or more rings, (e.g., phenyl, biphenyl, naphthyl, or the like, each ring either substituted or unsubstituted aromatic); arylalkyl having 1 to 3 separate or fused rings and from 6 to about 18 ring carbon atoms, with benzyl being an exemplary arylalkyl group; arylalkoxy having 1 to 3 separate or fused rings and from 6 to about 18 ring carbon atoms, with benzyloxy being an exemplary arylalkoxy group; or a saturated, unsaturated, or aromatic heterocyclic group having 1 to 3 separate or fused rings with 3 to about 8 members per ring and one or more N, O or S atoms, e.g. coumarinyl, quinolinyl, isoquinolinyl, quinazolinyl, pyridyl, pyrazinyl, pyrimidinyl, furanyl, pyrrolyl, thienyl, thiazolyl, triazinyl, oxazolyl, isoxazolyl, imidazolyl, indolyl, benzofuranyl, benzothiazolyl, tetrahydrofuranyl, tetrahydropyranyl, piperidinyl, morpholinyl, piperazinyl, and pyrrolidinyl. Such heterocyclic groups may be further substituted, e.g. with hydroxy, alkyl, alkoxy, halogen and amino.

A “patient” is a human or non-human animal in need of medical treatment. Medical treatment can include treatment of an existing condition, such as a disease or disorder, prophylactic or preventative treatment, or diagnostic treatment. In some embodiments the patient is a human patient.

“Pharmaceutically acceptable salts” of compounds of Formula I, and other active agents discussed herein also include solvates and hydrates of such active agents. The active agent may be modified by making non-toxic acid or base addition salts thereof. Examples of pharmaceutically acceptable salts include mineral or organic acid addition salts of basic residues such as amines; alkali or organic addition salts of acidic residues; and the like, and combinations comprising one or more of the foregoing salts. The pharmaceutically acceptable salts include non-toxic salts and the quaternary ammonium salts of compounds of Formula I or the at least one additional active agent. For example, non-toxic acid salts include those derived from inorganic acids such as hydrochloric, hydrobromic, sulfuric, sulfamic, phosphoric, nitric and the like; other acceptable inorganic salts include metal salts such as sodium salt, potassium salt, cesium salt, and the like; and alkaline earth metal salts, such as calcium salt, magnesium salt, and the like, and combinations comprising one or more of the foregoing salts. Pharmaceutically acceptable organic salts includes salts prepared from organic acids such as acetic, propionic, succinic, glycolic, stearic, lactic, malic, tartaric, citric, ascorbic, pamoic, maleic, hydroxymaleic, phenylacetic, glutamic, benzoic, salicylic, mesylic, esylic, besylic, sulfanilic, 2-acetoxybenzoic, fumaric, toluenesulfonic, methanesulfonic, ethane disulfonic, oxalic, isethionic, HOOC—(CH₂)ₙ—COOH where n is 0-4, and the like; organic amine salts such as triethylamine salt, pyridine salt, picoline salt, ethanolamine salt, triethanolamine salt, dicyclohexylamine salt, N,N′-dibenzylethylenediamine salt, and the like; and amino acid salts such as arginate, asparginate, glutamate, and the like; and combinations comprising one or more of the foregoing salts.

“Providing” means giving, administering, selling, distributing, transferring (for profit or not), manufacturing, compounding, or dispensing.

“Providing a compound of Formula I with at least one additional active agent” means the compound of Formula I and the additional active agent(s) are provided simultaneously in a single dosage form, provided concomitantly in separate dosage forms, or provided in separate dosage forms for administration separated by some amount of time that is within the time in which both the compound of Formula I and the at least one additional active agent are within the blood stream of a patient. The compound of Formula I and the additional active agent need not be prescribed for a patient by the same medical care worker. The additional active agent or agents need not require a prescription. Administration of the compound of Formula I or the at least one additional active agent can occur via any appropriate route, for example, oral tablets, oral capsules, oral liquids, inhalation, injection, suppositories or topical contact.

“Treatment,” as used herein includes providing a compound of Formula I and at least one additional active agent sufficient to: (a) prevent a disease or a symptom of a disease from occurring in a patient who may be predisposed to the disease but has not yet been diagnosed as having it (e.g. including diseases that may be associated with or caused by a primary disease (as in liver fibrosis that can result in the context of chronic HCV infection); (b) inhibiting the disease, i.e. arresting its development; and (c) relieving the disease, i.e., causing regression of the disease. “Treating” and “treatment” also means providing a therapeutically effective amount of a compound of Formula I and at least one additional active agent to a patient having or susceptible to a hepatitis C infection. A “therapeutically effective amount” of a pharmaceutical combination of this invention means an amount effective, when administered to a patient, to provide a therapeutic benefit such as an amelioration of symptoms, e.g., an amount effective to decrease the symptoms of a hepatitis C infection. For example a patient infected with a hepatitis C virus may present elevated levels of certain liver enzymes, including AST and ALT. Normal levels of AST are from 5 to 40 units per liter of serum (the liquid part of the blood) and normal levels of ALT are from 7 to 56 units per liter of serum. A therapeutically effect amount is thus an amount sufficient to provide a significant reduction in elevated AST and ALT levels or an amount sufficient to provide a return of AST and ALT levels to the normal range. A therapeutically effective amount is also an amount sufficient to prevent a significant increase or significantly reduce the detectable level of virus or viral antibodies in the patient's blood, serum, or tissues. One method of determining treatment efficacy includes measuring HCV RNA levels by a convention method for determining viral RNA levels such as the Roch TaqMan assay. In certain preferred embodiments treatment reduces HCV RNA levels below the limit of quantitation (30 IU/mL, as measured by the Roche TaqMan(R) assay) or more preferably below the limit of detection (10 IU/mL, Roche TaqMan).

A significant increase or reduction in the detectable level of virus or viral antibodies is any detectable change that is statistically significant in a standard parametric test of statistical significance such as Student's T-test, where p<0.05.

### Pharmaceutical Combinations

The invention also provides pharmaceutical combinations comprising a compound of Formula I (as shown above) or a pharmaceutical acceptable salt thereof and at least one additional active agent. The compound of Formula I may be provided in a single dosage form, or may be formulated as separate dosage forms and provided together as separate dosage forms. For example the compound of Formula I and the at least one additional active agent may be provided together in a container as a packaged pharmaceutical combination. Such packaged combinations may contain instructions for using the combination to treat a hepatitis C infection.

Combinations providing either synergistic or additive effects are useful in treating HCV and are included in the invention. In certain embodiments, the compound of Formula I and the at least one of the additional active agent provide a synergistic effect. In other embodiments the compound of Formula I and the at least one additional active agent provide an additive effect. A combination that provides a synergistic effect is a combination that provides a greater therapeutic or prophylactic effect than the incremental improvement in treatment outcome that could be predicted or expected from a additive combination of (i) the therapeutic or prophylactic benefit of the compound of Formula I when administered at that same dosage as a monotherapy and (ii) the therapeutic or prophylactic benefit of the additional active agent when administered at the same dosage as a monotherapy. For example a combination that exhibits a combination index (CI) equal or less than 0.9 in an assay for synergistic drug effects as calculated by the Chou and Talalay method is considered a synergistic combination.

The at least one additional active agent may have direct antiviral activity or may act via some other mechanism to improve treatment efficacy. For example NS3/4A protease inhibitors, including VX-950 are metabolized by cyclochrome p450 enzymes, particularly the 3A4 isozyme. Efficacy of anti-viral therapy can be increased by inhibiting the CYP isozymes which metabolize the anti-viral drug, thus improving patient exposure to the drug. Cytochrome p450 monooxygenase inhibitors are thus useful as additional active agents in the methods and pharmaceutical combinations of this invention.

Particular compounds of Formula I useful in the pharmaceutical combinations described herein include the following compounds and their pharmaceutically acceptable salts, hydrates, prodrugs, and polymorphs of these compounds:
1-(3-cyclopropyl-4-(pentyloxy)phenyl)-3-nicotinoylthiourea;
methyl 2-oxo-2-(5-((3-(4-(pentyloxy)-3-(trifluoromethyl)phenyl)thioureido)carbonyl)pyridin-2-ylamino)acetate;
methyl 2-(5-((3-(4-(heptyloxy)phenyl)thioureido)carbonyl)pyridin-2-ylamino)-2-oxoacetate;
1-(2-(2-amino-2-oxoacetamido)nicotinoyl)-3-(4-(pentyloxy)-3-(trifluoromethyl)phenyl)thiourea;
1-(6-(2-(dimethylamino)ethoxy)nicotinoyl)-3-(4-(pentyloxy)-3-(trifluoromethyl)phenyl)thiourea;
1-(2-methylnicotinoyl)-3-(4-(pentyloxy)-3-(trifluoromethyl)phenyl)thiourea;
1-(6-(4-methylpiperazin-1-yl)nicotinoyl)-3-(4-pentylphenyl)thiourea;
1-(6-(4-methylpiperazin-1-yl)nicotinoyl)-3-(4-(pentyloxy)phenyl)thiourea;
1-(3-fluoro-4-(pentyloxy)phenyl)-3-(6-(4-methylpiperazin-1-yl)nicotinoyl)thiourea;
1-(6-(4-methylpiperazin-1-yl)nicotinoyl)-3-(4-(pentyloxy)-3-(trifluoromethyl)phenyl)thiourea;
1-(3-methyl-4-(pentyloxy)phenyl)-3-(6-(4-methylpiperazin-1-yl)nicotinoyl)thiourea;
1-(6-hydroxynicotinoyl)-3-(4-(pentyloxy)phenyl)thiourea;
1-(6-hydroxynicotinoyl)-3-(4-pentylphenyl)thiourea;
1-(3-fluoro-4-(pentyloxy)phenyl)-3-(6-hydroxynicotinoyl)thiourea;
1-(6-hydroxynicotinoyl)-3-(4-(pentyloxy)-3-(trifluoromethyl)phenyl)thiourea;
1-(5-hydroxynicotinoyl)-3-(4-(pentyloxy)-3-(trifluoromethyl)phenyl)thiourea;
1-(3-fluoro-4-(pentyloxy)phenyl)-3-(5-hydroxynicotinoyl)thiourea;
1-(6-(morpholine-4-carbonyl)nicotinoyl)-3-(4-(pentyloxy)-3-(trifluoromethyl)phenyl)thiourea;
1-(6-((dimethylamino)methyl)nicotinoyl)-3-(4-(pentyloxy)-3-(trifluoromethyl)phenyl)thiourea;
1-(6-acetamidonicotinoyl)-3-(4-(pentyloxy)-3-(trifluoromethyl)phenyl)thiourea;
(5-((3-(4-(pentyloxy)-3-(trifluoromethyl)phenyl)thioureido) carbonyl)pyridin-2-yl)methyl acetate;
1-(6-(hydroxymethyl)nicotinoyl)-3-(4-(pentyloxy)-3-(trifluoromethyl)phenyl)thiourea;
1-(6-(morpholinomethyl)nicotinoyl)-3-(4-(pentyloxy)phenyl)thiourea;
1-(6-(morpholinomethyl)nicotinoyl)-3-(4-(pentyloxy)-3-(trifluoromethyl)phenyl)thiourea;
1-(6-((dimethylamino)methyl)nicotinoyl)-3-(4-pentylphenyl)thiourea;
1-(4-(heptyloxy)phenyl)-3-(6-(morpholine-4-carbonyl)nicotinoyl)thiourea;
1-(6-(morpholine-4-carbonyl)nicotinoyl)-3-(4-(octyloxy)phenyl)thiourea;
1-(6-(morpholine-4-carbonyl)nicotinoyl)-3-(4-pentylphenyl)thiourea;
1-(4-(heptyloxy)phenyl)-3-(6-((4-methylpiperazin-1-yl)methyl)nicotinoyl)thiourea;
1-(6-((4-methylpiperazin-1-yl)methyl)nicotinoyl)-3-(4-(pentyloxy)-3-(trifluoromethyl)phenyl)thiourea;
1-(6-((dimethylamino)methyl)nicotinoyl)-3-(4-(pentyloxy)phenyl)thiourea;
1-(4-(heptyloxy)phenyl)-3-(6-hydroxynicotinoyl)thiourea;
1-(6-((dimethylamino)methyl)nicotinoyl)-3-(4-(hexyloxy)phenyl)thiourea;
1-(4-(hexyloxy)phenyl)-3-(6-(morpholinomethyl)nicotinoyl)thiourea;
1-(4-(heptyloxy)phenyl)-3-(6-(morpholinomethyl)nicotinoyl) thiourea;
1-(3-fluoro-4-(pentyloxy)phenyl)-3-(6-morpholinonicotinoyl)thiourea;
1-(6-cyanonicotinoyl)-3-(4-(pentyloxy)-3-(trifluoromethyl)phenyl)thiourea;
1-(6-cyanonicotinoyl)-3-(3-fluoro-4-(pentyloxy)phenyl)thiourea;
1-(2-cyanonicotinoyl)-3-(4-(pentyloxy)phenyl)thiourea;
1-(6-(2-morpholinoethoxy)nicotinoyl)-3-(4-(pentyloxy)-3-(trifluoromethyl)phenyl)thiourea;
1-(3-fluoro-4-(pentyloxy)phenyl)-3-(6-(2-morpholinoethoxy)nicotinoyl)thiourea;
1-(6-(2-morpholinoethoxy)nicotinoyl)-3-(4-(pentyloxy)phenyl)thiourea;
1-(6-(2-morpholinoethoxy)nicotinoyl)-3-(4-pentylphenyl)thiourea;
1-(6-(dimethylamino)nicotinoyl)-3-(4-(pentyloxy)-3-(trifluoromethyl)phenyl)thiourea;
1-(6-(dimethylamino)nicotinoyl)-3-(4-(pentyloxy)phenyl)thiourea;
1-(6-(dimethylamino)nicotinoyl)-3-(4-pentylphenyl)thiourea;
1-(5-(furan-3-yl)nicotinoyl)-3-(4-(pentyloxy)-3-(trifluoromethyl)phenyl)thiourea;
1-(3-fluoro-4-(pentyloxy)phenyl)-3-(5-(furan-3-yl)nicotinoyl)thiourea;
1-(5-(furan-3-yl)nicotinoyl)-3-(4-(pentyloxy)phenyl)thiourea;
1-(3-cyano-4-(pentyloxy)phenyl)-3-nicotinoylthiourea;
1-(5-bromonicotinoyl)-3-(4-(pentyloxy)-3-(trifluoromethyl)phenyl)thiourea;
1-(6-(dimethylcarbamoyl)nicotinoyl)-3-(4-(pentyloxy)-3-(trifluoromethyl)phenyl)thiourea;
1-(6-(dimethylcarbamoyl)nicotinoyl)-3-(3-fluoro-4-(pentyloxy)phenyl)thiourea;
1-(3-cyano-4-(pentyloxy)phenyl)-3-(6-(dimethylcarbamoyl)nicotinoyl)thiourea;
1-(6-(dimethylcarbamoyl)nicotinoyl)-3-(4-(pentyloxy)phenyl)thiourea;
1-(3-chloro-4-(pentyloxy)phenyl)-3-(6-cyanonicotinoyl)thiourea;
1-(5-cyanonicotinoyl)-3-(4-(pentyloxy)-3-(trifluoromethyl)phenyl)thiourea;
methyl 5-((3-(4-(pentyloxy)-3-(trifluoromethyl)phenyl)thioureido)carbonyl)nicotinate;
1-(3-fluoro-4-(pentyloxy)phenyl)-3-(6-(1-methylpiperazine-4-carbonyl)nicotinoyl)thiourea;
1-(3-chloro-4-(pentyloxy)phenyl)-3-(6-(1-methylpiperazine-4-carbonyl)nicotinoyl)thiourea;
1-(6-(1-methylpiperazine-4-carbonyl)nicotinoyl)-3-(4-(pentyloxy)phenyl)thiourea;
1-(3-chloro-4-(pentyloxy)phenyl)-3-nicotinoylthiourea;
1-(4-(pentyloxy)phenyl)-3-(6-(2-(pyrrolidin-1-yl)ethyl)nicotinoyl)thiourea;
1-(4-pentylphenyl)-3-(6-(2-(pyrrolidin-1-yl)ethyl)nicotinoyl)thiourea;
1-(2-(4-fluorophenoxy)nicotinoyl)-3-(4-pentylphenyl)thiourea;
1-nicotinoyl-3-(4-(octyloxy)phenyl)thiourea;
1-nicotinoyl-3-(4-octylphenyl)thiourea;
1-(4-(hexyloxy)phenyl)-3-nicotinoylthiourea;
1-(4-hexylphenyl)-3-nicotinoylthiourea;
1-(3-fluoro-4-(pentyloxy)phenyl)-3-nicotinoylthiourea;
1-nicotinoyl-3-(4-(pentyloxy)-3-(trifluoromethyl)phenyl)thiourea;
1-(4-butoxyphenyl)-3-nicotinoylthiourea;
1-(4-(heptyloxy)-3-(trifluoromethyl)phenyl)-3-nicotinoylthiourea;
1-(3-chloro-4-(pentyloxy)phenyl)-3-nicotinoylthiourea; or
1-(4-isopropoxy-3-(trifluoromethyl)phenyl)-3-nicotinoylthiourea.

Compounds of Formula I useful in the pharmaceutical combinations described herein also include following compounds and the pharmaceutically acceptable salts, hydrates, prodrugs, and polymorphs of these compounds:

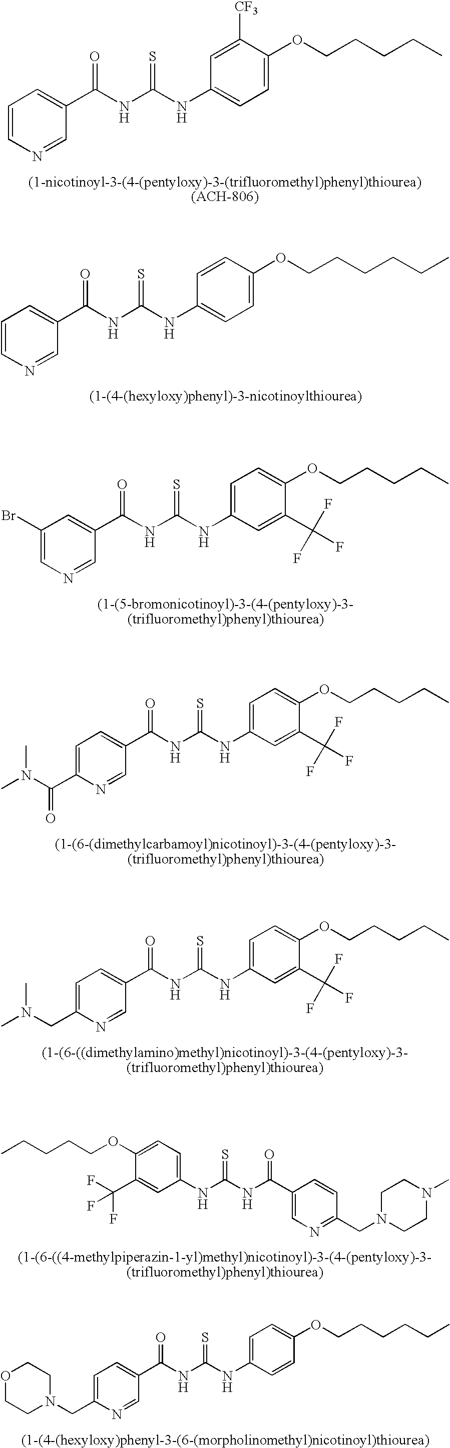

The additional active agent or active agents in the methods of treatment and pharmaceutical combinations provided herein may be a caspase inhibitor, cyclophilin inhibitor, cytochrome P450 monooxygenase inhibitor, a glucocorticoid, a hematopoietin, a fusion inhibitor, an entry inhibitor, a capsid inhibitor, a helicase inhibitor (NS3), a homeopathic therapeutic agent, an immunomodulatory compound (includes interferon), an immunosuppressant, an interleukin, an interferon enhancer, an IRES inhibitor, a monoclonal or polyclonal antibody, a nucleoside analogue, a non-nucleoside inhibitor, a P7 protein inhibitor, a polymerase inhibitor (including RNA-dependent RNA polymerase NS5B), protease inhibitors (including inhibitors of the HCV metalloprotease NS2-3 and NS3/4A protease inhibitors), RNA interference agent, a therapeutic vaccine, a TNF agonist, a tubulin inhibitor, NS5A inhibitor, NS4B inhibitor, a kinase inhibitor, a Toll like receptor agonist or a sphingosine-1-phosphate receptor modulator.

Without being bound by a particular theory it is believed that ACH-806 and other compounds of Formula I inhibit HCV replication through effects at HCV NS4A. In most embodiments the additional active agent or agents will have a mechanism of action distinct from that of ACH-806.

In certain embodiments the additional active agent (or agents) is an HCV protease inhibitor or HCV polymerase inhibitor. For example the protease inhibitor may be telaprevir (VX-950) and the polymerase inhibitor may be valopicitabine, or NM 107, the active agent into which valopicitabine is converted.

The methods of treatment described herein include at least a compound of Formula I and a second active agent, but may also include additional active agents. In certain embodiments method of treatment includes providing a patient with a compound of Formula I and an interferon such as a pegylated interferon or interferon gamma. The interferon may be the only compound provided with the compound of Formula I or may be provided with an additional active agent that is not an interferon.

The invention methods of treatment and pharmaceutical combinations including compounds of Formula I including any of the following compounds and substances as an additional active agent:

Caspase inhibitors: IDN 6556 (Idun Pharmaceuticals)

Cyclophilin Inhibitors: NIM811 (Novartis) and DEBIO-025 (Debiopharm) Cytochrome P450 monooxygenase inhibitors: ritonavir (WO 94/14436), ketoconazole, troleandomycin, 4-methylpyrazole, cyclosporin, clomethiazole, cimetidine, itraconazole, fluconazole, miconazole, fluvoxamine, fluoxetine, nefazodone, sertraline, indinavir, nelfinavir, amprenavir, fosamprenavir, saquinavir, lopinavir, delavirdine, erythromycin, VX-944, and VX-497. Preferred CYP inhibitors include ritonavir, ketoconazole, troleandomycin, 4-methylpyrazole, cyclosporin, and clomethiazole

Glucocorticoids: hydrocortisone, cortisone, prednisone, prednisolone, methylprednisolone, triamcinolone, paramethasone, betamethasone, and dexamethasone

Hematopoietins: hematopoietin-1 and hematopoietin-2. Other members of the hematopoietin superfamily such as the various colony stimulating factors (e.g. (e.g. G-CSF, GM-CSF, M-CSF), Epo, and SCF (stem cell factor)

Homeopathic Therapies Milk Thistle, silymarin, ginseng, glycyrrhizin, licorice root, schisandra, vitamin C, vitamin E, beta carotene, and selenium

Immunomodulatory compounds: thalidomide, IL-2, hematopoietins, IMPDH inhibitors, for example Merimepodib (Vertex Pharmaceuticals Inc.), interferon, including natural interferon (such as OMNIFERON, Viragen and SUMIFERON, Sumitomo, a blend of natural interferons), natural interferon alpha (ALFERON, Hemispherx Biopharma, Inc.), interferon alpha n1 from lymphblastoid cells (WELLFERON, Glaxo Wellcome), oral alpha interferon, Peg-interferon, Peg-interferon alfa 2a (PEGASYS, Roche), recombinant interferon alfa 2a (ROFERON, Roche), inhaled interferon alpha 2b (AERX, Aradigm), Peg-interferon alpha 2b (ALBUFERON, Human Genome Sciences/Novartis, PEGINTRON, Schering), recombinant interferon alfa 2b (INTRON A, Schering), pegylated interferon alfa 2b (PEG-INTRON, Schering, VIRAFERONPEG, Schering), interferon beta-1a (REBIF, Serono, Inc. and Pfizer), consensus interferon alpha (INFERGEN, Valeant Pharmaceutical), interferon gamma-1b (ACTIMMUNE, Intermune, Inc.), un-pegylated interferon alpha, alpha interferon, and its analogs, and synthetic thymosin alpha 1 (ZADAXIN, SciClone Pharmaceuticals Inc.)

Immunosupressants: sirolimus (RAPAMUNE, Wyeth)

Interleukins: (IL-1, IL-3, IL-4, IL-5, IL-6, IL-10, IL-11, IL-12), LIF, TGF-beta, TNF-alpha) and other low molecular weight factors (e.g. AcSDKP, pEEDCK, thymic hormones, and minicytokines)

Interferon Enhancers: EMZ702 (Transition Therapeutics)

IRES inhibitors: VGX-410C (VGX Pharma)

Monoclonal and Polyclonal antibodies: XTL-6865 (XTL), HuMax-HepC (Genmab), Hepatitis C Immune Globin (human) (CIVACIR, Nabi Biopharmaceuticals)

Nucleoside analogues: Lamivudine (EPIVIR, 3TC, GlaxoSmithKline), MK-0608 (Merck), zalcitabine (HIVID, Roche US Pharmaceuticals), ribavirin (including COPEGUS (Roche), REBETOL (Schering), VILONA (ICN Pharmaceuticals, and VIRAZOLE (ICN Pharmaceuticals), and viramidine (Valeant Pharmaceuticals), an amidine prodrug of ribavirin. Combinations of nucleoside analogues may also be employed.

Non-nucleoside inhibitors: delaviridine (RESCRIPTOR, Pfizer), and HCV-796 (Viropharm)

P7 protein inhibitor: amantadine (SYMMETREL, Endo Pharmaceuticals, Inc.)

Polymerase inhibitors: NM283 (valopicitabine) (Idenix) and NM 107 (Idenix), PSI-6130 (Roche/Pharmasset).

Protease inhibitors: BILN-2061 (Boehringer Ingelheim), GW-433908 (prodrug of Amprenavir, Glaxo/Vertex), indinavir (CRIXWAN, Merck), ITMN-191 (Intermune/Array Biopharma), VX950 (Vertex) and combinations comprising one or more of the foregoing protease inhibitors

RNA interference: SIRNA-034 RNAi (Sima Therapeutics)

Therapeutic Vaccines: IC41 (Intercell), IMN-0101 (Imnogenetics), GI 5005 (Globeimmune), Chronvac-C (Tripep/Inovio), ED-002 (Imnogenetics), Hepavaxx C (ViRex Medical)

TNF agonists: adalimumab (HUMIRA, Abbott), entanercept (ENBREL, Amgen and Wyeth), infliximab (REMICADE, Centocor, Inc.)

Tubulin inhibitors: Colchicine

Sphingosine-1-phosphate receptor modulators: FTY720 (Novartis)

TLR agonists: ANA-975 (Anadys Pharmaceuticals), TLR7 agonist (Anadys Pharmaceuticals), CPG10101 (Coley), and TLR9 agonists including CPG 7909 (Coley)

Cyclophilin Inhibitors: NIM811 (Novartis) and DEBIO-025 (Debiopharm)

Patients receiving hepatitis C medications are typically given interferon together with another active agent. Thus methods of treatment and pharmaceutical combinations in which a compound of Formula I is provided together with an interferon, such as pegylated interferon alfa 2a, as the additional active agents are included as embodiments. Similarly methods and pharmaceutical combinations in which ribarvirin is an additional active agent are provided herein. and as embodiments.

### Pharmaceutical Formulations

While it is possible for the active agents present in the methods and the pharmaceutical combinations described herein, including compounds of Formula I, to be administered neat, it may be preferable to formulate the active agents as pharmaceutical formulations. Pharmaceutical formulations may be prepared with pharmaceutically acceptable excipients such as carriers, solvents, stabilizers, adjuvants, diluents, glidants, etc., depending upon the particular mode of administration and dosage form. Formulations optionally contain excipients such as those set forth in the Handbook of Pharmaceutical Excipients, 5^{th} Edition (2005).

Active agents useful in the methods and pharmaceutical formulations described herein may be formulated in a unitary dosage form, or in separate dosage forms intended for simultaneous or sequential administration to a patient in need of treatment. When administered sequentially, the combination may be administered in two or more, three or more, four or more, five or more, or six or more administrations. In an alternative embodiment, it is possible to administer one or more compounds of the present invention and one or more additional active ingredients by different routes.

Pharmaceutical formulations of the invention comprise a therapeutically or prophylactically effective amount of a compound of Formula I and an additional active agent, together with one or more pharmaceutically acceptable excipients. A therapeutically or prophylactically effective amount of a combination of the invention includes a viral inhibitory amount of the combination.

Combinations of the invention may be administered by any route appropriate to the condition to be treated. Suitable routes include parenteral (including subcutaneous, intramuscular, intravenous, intradermal, intrathecal and epidural), oral, nasal, topical (including buccal and sublingual), rectal, vaginal, and the like. It will be appreciated that the preferred route of administration may vary, depending for example upon the condition of the recipient and the duration of the treatment. In a preferred embodiment, treatment is administered orally or parenterally to a patient who has antibodies to hepatitis C virus.

Formulations of the present invention, e.g., for parenteral or oral administration, are most typically solids, liquid solutions, emulsions or suspensions, while inhalable formulations for pulmonary administration are generally liquids or powders, with powder formulations being generally preferred. A pharmaceutical combination of the invention may also be formulated as a lyophilized solid that is reconstituted with a physiologically-compatible solvent prior to administration. Alternative pharmaceutical formulations of the invention may be prepared as syrups, elixirs, creams, ointments, tablets, and the like.

Formulations for oral use include, for example, tablets, troches, lozenges, electuaries, aqueous or oil suspensions, non-aqueous solutions, dispersible powders or granules (including micronized particles or nanoparticles), emulsions, hard or soft capsules, syrups or elixirs may be prepared. Formulations intended for oral use may be prepared according to any method known to the art for the manufacture of pharmaceutical formulations, and such formulations may contain one or more agents including sweetening agents, flavoring agents, coloring agents and preserving agents, in order to provide a palatable preparation.

Pharmaceutical formulations will contain excipients. Suitable excipients may be carrier molecules that include large, slowly metabolized macromolecules such as proteins, polysaccharides, polylactic acids, polyglycolic acids, polymeric amino acids, amino acid copolymers, and inactive virus particles. Other exemplary excipients include antioxidants such as ascorbic acid; chelating agents such as EDTA; carbohydrates such as dextrin, hydroxyalkylcellulose, hydroxyalkylmethylcellulose, stearic acid; liquids such as oils, water, saline, glycerol and ethanol; wetting or emulsifying agents; pH buffering substances; and the like. Liposomes are also included within the definition of pharmaceutically acceptable excipients.

Pharmaceutically acceptable excipients particularly suitable for use in conjunction with tablets include, for example, inert diluents, such as celluloses, calcium or sodium carbonate, lactose, calcium or sodium phosphate; disintegrating agents, such as croscarmellose sodium, cross-linked povidone, maize starch, or alginic acid; binding agents, such as povidone, starch, gelatin or acacia; and lubricating agents, such as magnesium stearate, stearic acid or talc.

Carriers are one class of excipients that will frequently be used in the combinations described herein. Carrier material necessary to produce a single dosage form will be determined by the skilled artisan and will vary depending upon considerations including the host, the nature of the condition being treated, the particular mode of administration, the pharmaceutical formulation, and the toxicity. Active agents may be formulated with an appropriate and convenient amount of carrier material, which may vary, for example from about 5% to about 95% of the total composition (weight:weight).

Tablets may be uncoated or may be coated by known techniques including microencapsulation to delay disintegration and adsorption in the gastrointestinal tract and thereby provide a sustained action over a longer period. For example, a time delay material such as glyceryl monostearate or glyceryl distearate alone or with a wax may be employed.

Formulations for oral use may be also presented as hard gelatin capsules where the active ingredient is mixed with an inert solid diluent, for example celluloses, lactose, calcium phosphate or kaolin, or as soft gelatin capsules wherein the active ingredient is mixed with non-aqueous or oil medium, such as glycerin, propylene glycol, polyethylene glycol, peanut oil, liquid paraffin or olive oil.

In another embodiment, pharmaceutical formulations are formulated as suspensions comprising a compound of the present invention in an admixture with at least one pharmaceutically acceptable excipient suitable for the manufacture of a suspension. In yet another embodiment, pharmaceutical formulations are formulated as dispersible powders and granules suitable for preparation of a suspension by the addition of suitable excipients. Excipients suitable for use in connection with suspensions include suspending agents, such as sodium carboxymethylcellulose, methylcellulose, hydroxypropyl methylcelluose, sodium alginate, polyvinylpyrrolidone, gum tragacanth, gum acacia, dispersing or wetting agents such as a naturally occurring phosphatide (e.g., lecithin), a condensation product of an alkylene oxide with a fatty acid (e.g., polyoxyethylene stearate), a condensation product of ethylene oxide with a long chain aliphatic alcohol (e.g., heptadecaethyleneoxycethanol), a condensation product of ethylene oxide with a partial ester derived from a fatty acid and a hexitol anhydride (e.g., polyoxyethylene sorbitan monooleate); and thickening agents, such as carbomer, beeswax, hard paraffin or cetyl alcohol. The suspensions may also contain one or more preservatives such as acetic acid, methyl and/or n-propyl p-hydroxy-benzoate; one or more coloring agents; one or more flavoring agents; and one or more sweetening agents such as sucrose or saccharin.

Oil suspensions may be formulated by suspending the active ingredient in a vegetable oil, such as arachis oil, olive oil, sesame oil or coconut oil, or in a mineral oil such as liquid paraffin. The oral suspensions may contain a thickening agent, such as beeswax, hard paraffin or cetyl alcohol. Sweetening agents and flavoring agents optionally may be added to provide a palatable oral preparation. One or more antioxidant, such as ascorbic acid, for example, may be added as a preservative.

The pharmaceutical formulations of the invention may also be in the form of oil-in-water emulsions. In an aspect, the oily phase of an emulsion may comprise only one or more emulsifiers (otherwise known as emulgents). In a preferred aspect, the oily phase comprises a mixture of at least one emulsifier with a fat or an oil or with both a fat and an oil. Preferably, a hydrophilic emulsifier is included together with a lipophilic emulsifier, which acts as a stabilizer. It is also preferred to include both an oil and a fat. Emulgents and emulsion stabilizers suitable for use in the formulation of the invention include Tween® 60, Span® 80, cetostearyl alcohol, benzyl alcohol, myristyl alcohol, glyceryl mono-stearate and sodium lauryl sulfate. In an aspect of the present invention, the oily phase comprises a vegetable oil, such as olive oil or arachis oil, a mineral oil, such as liquid paraffin, or a mixture of these. Suitable emulsifying agents include naturally-occurring gums, such as gum acacia and gum tragacanth; naturally occurring phosphatides, such as soybean lecithin, esters or partial esters derived from fatty acids; hexitol anhydrides, such as sorbitan monooleate; and condensation products of these partial esters with ethylene oxide, such as polyoxyethylene sorbitan monooleate. The emulsion may also contain sweetening and flavoring agents. Syrups and elixirs may be formulated with sweetening agents, such as glycerol, sorbitol or sucrose. In an aspect, such formulations may also contain a demulcent, a preservative, a flavoring, a coloring agent, or any combination of these ingredients. An emulsion or suspension may be formulated according to the known art using those suitable dispersing or wetting agents and suspending agents.

In some embodiments, the pharmaceutical formulations are in the form of a sterile injectable preparation. An injectable may be administered for example by injection, infusion, or as a bolus. Injectable preparations include by way of non-limiting example sterile injectable aqueous emulsions and oleaginous suspensions. The sterile injectable preparation may also be a sterile injectable solution or suspension in a non-toxic parenterally acceptable diluent or solvent, such as a solution in 1,2-propane-diol. The sterile injectable preparation may also be prepared as a lyophilized powder. Among the acceptable vehicles and solvents that may be employed are water, Ringer's solution, and isotonic sodium chloride solution. In addition, sterile fixed oils may be employed as a solvent or suspending medium. For this purpose any bland fixed oil may be employed including synthetic mono- or di-glycerides. In addition, fatty acids such as oleic acid may likewise be used in the preparation of injectables.

Active agents of the pharmaceutical combinations described herein may be formulated for oral administration in a lipid-based formulation suitable for low solubility compounds. Lipid-based formulations can generally enhance the oral bioavailability of such compounds. In an embodiment, a pharmaceutical formulation of the invention comprises a therapeutically or prophylactically effective amount of a compound of the present invention, together with at least one pharmaceutically acceptable excipient selected from: medium chain fatty acids or propylene glycol esters thereof (e.g., propylene glycol esters of edible fatty acids such as caprylic and capric fatty acids) and pharmaceutically acceptable surfactants such as polyoxyl 40 hydrogenated castor oil. Cyclodextrins may be added as aqueous solubility enhancers. Cyclodextrins include hydroxypropyl, hydroxyethyl, glucosyl, maltosyl and maltotriosyl derivatives of α-, β-, and γ-cyclodextrin. A particularly preferred cyclodextrin solubility enhancer is hydroxypropyl-β-cyclodextrin (HPBC), which may be added to any of the above-described formulations to further improve the aqueous solubility characteristics of the compounds of the present invention. In one embodiment, the composition comprises 0.1% to 20% hydroxypropyl-β-cyclodextrin, more preferably 1% to 15% hydroxypropyl-β-cyclodextrin, and even more preferably from 2.5% to 10% hydroxypropyl-β-cyclodextrin. The amount of solubility enhancer employed will depend on the amount of the compound of the present invention in the composition.

The formulations of the present invention may be provided in unit dosage form or in multi-dose containers, including for example sealed ampoules and vials, and may be stored in a freeze-dried or lyophilized condition, requiring only the addition of the sterile liquid carrier, for example saline for injection, immediately prior to use. In an embodiment, unit dosage formulations contain a daily dose or subdose, or a fraction thereof, of the active ingredient.

### Packaged Formulations

The invention includes packaged pharmaceutical combinations. Such packaged combinations include a compound of Formula I and at least one additional active agent together in a container. The container may additionally include instructions for using the combination to treat or prevent a hepatitis C infection in a patient.

The compound of Formula I and the one or more additional active agent may be combined in a single dosage form or may be present in separate dosage forms.

### Methods of Treatment

The invention includes methods of preventing and treating hepatitis C infections, by administering an effective amount of a more compounds of Formula I and at least one additional active agent to patient at risk for hepatitis C infection or infected with a hepatitis C virus.

According to the methods of the invention, the combination of active agents may be: (1) co-formulated and administered or delivered simultaneously in a combined formulation; (2) delivered by alternation or in parallel as separate formulations; or (3) by any other combination therapy regimen known in the art. When delivered in alternation therapy, the methods of the invention may comprise administering or delivering the active ingredients sequentially, e.g., in separate solution, emulsion, suspension, tablets, pills or capsules, or by different injections in separate syringes. In general, during alternation therapy, an effective dosage of each active ingredient is administered sequentially, i.e., serially, whereas in simultaneous therapy, effective dosages of two or more active ingredients are administered together. Various sequences of intermittent combination therapy may also be used.

The pharmaceutical combinations disclosed herein are useful for preventing and treating hepatitis C infections in patients. An effective amount of a pharmaceutical combination of the invention may be an amount sufficient to (a) prevent hepatitis C or a symptom of a hepatitis C from occurring in a patient who may be predisposed to hepatitis C but has not yet been diagnosed as having it or prevent diseases that may be associated with or caused by a primary hepatitis C infection (such as liver fibrosis that can result in the context of chronic HCV infection); (b) inhibit the progression of hepatitis C; and (c) cause a regression of the hepatitis C infection. An amount of a pharmaceutical composition effect to inhibit the progress or cause a regression of hepatitis C includes an amount effective to stop the worsening of symptoms of hepatitis C or reduce the symptoms experienced by a patient infected with the hepatitis C virus. Alternatively a halt in progression or regression of hepatitis C may be indicated by any of several markers for the disease. For example, a lack of increase or reduction in the hepatitis C viral load or a lack of increase or reduction in the number of circulating HCV antibodies in a patient's blood are markers of a halt in progression or regression of hepatitis C infection. Other hepatitis C disease markers include aminotransferase levels, particularly levels of the liver enzymes AST and ALT. Normal levels of AST are from 5 to 40 units per liter of serum (the liquid part of the blood) and normal levels of ALT are from 7 to 56 units per liter of serum. These levels will typically be elevated in a HCV infected patient. Disease regression is usually marked by the return of AST and ALT levels to the normal range.

Symptoms of hepatitis C that may be affected by an effective amount of a pharmaceutical combination of the invention include decreased liver function, fatigue, flu-like symptoms: fever, chills, muscle aches, joint pain, and headaches, nausea, aversion to certain foods, unexplained weight loss, psychological disorders including depression, tenderness in the abdomen, and jaundice.

“Liver function” refers to a normal function of the liver, including, but not limited to, a synthetic function including synthesis of proteins such as serum proteins (e.g., albumin, clotting factors, alkaline phosphatase, aminotransferases (e.g., alanine transaminase, aspartate transaminase), 5′-nucleosidase, y glutaminyltranspeptidase, etc.), synthesis of bilirubin, synthesis of cholesterol, and synthesis of bile acids; a liver metabolic function, including carbohydrate metabolism, amino acid and ammonia metabolism, hormone metabolism, and lipid metabolism; detoxification of exogenous drugs; and a hemodynamic function, including splanchnic and portal hemodynamics.

An effective amount of a combination described herein will also provide a sufficient concentration of the active agents in the concentration when administered to a patient. A sufficient concentration of an active agent is a concentration of the agent in the patient's body necessary to prevent or combat the infection. Such an amount may be ascertained experimentally, for example by assaying blood concentration of the agent, or theoretically, by calculating bioavailability. The amount of an active agent sufficient to inhibit viral infection in vitro may be determined with a conventional assay for viral infectivity such as a replicon based assay, which has been described in the literature.

The invention also includes using pharmaceutical combinations comprising a compound of Formula I and at least one additional active agent in prophylactic therapies. In the context of prophylactic or preventative treatment an effective amount of a compound of the invention is an amount sufficient to significantly decrease the patient's risk of contracting a hepatitis C infection.

Methods of treatment include providing certain dosage amounts of the compound of Formula I and the at least one additional active agent to a patient. Dosage levels of each active agent of from about 0.1 mg to about 140 mg per kilogram of body weight per day are useful in the treatment of the above-indicated conditions (about 0.5 mg to about 7 g per patient per day). The amount of active ingredient that may be combined with the carrier materials to produce a single dosage form will vary depending upon the patient treated and the particular mode of administration. Dosage unit forms will generally contain between from about 1 mg to about 500 mg of each active agent. In certain embodiments 25 mg to 500 mg, or 25 mg to 200 mg of ACH-806 or other compound of Formula I are provided daily to a patient and an effective amount of at least one additional active agent is also provided. When the additional active agent is NM 283 (valopicitabine), 100 mg to 1000 mg/day, or 200 mg to 800 mg/day, or 200 to 400 mg/day of either of those agents are typically provided to the patient. When the additional active agent is VX-950, 1000 mg to 3750 mg/day, or 1200 mg to 1800 mg/day are administered to the patient. Treatment regiments in which VX-950 is an additional active agent and about 350 to about 450 mg or about 700 to about 800 mg of VX-950 are administered to a patient three times per day or about 350 to about 450 mg or about 700 to about 800 mg is administered every 12 hours are particularly included in the invention.

Frequency of dosage may also vary depending on the compound used and the particular disease treated. However, for treatment of most infectious disorders, a dosage regimen of 4 times daily or less is preferred and a dosage regimen of 1 or 2 times daily is particularly preferred.

It will be understood, however, that the specific dose level for any particular patient will depend upon a variety of factors including the activity of the specific compound employed, the age, body weight, general health, sex, diet, time of administration, route of administration, and rate of excretion, drug combination and the severity of the particular disease undergoing therapy.

### EXAMPLES

### Example 1

### Preparation of Compounds

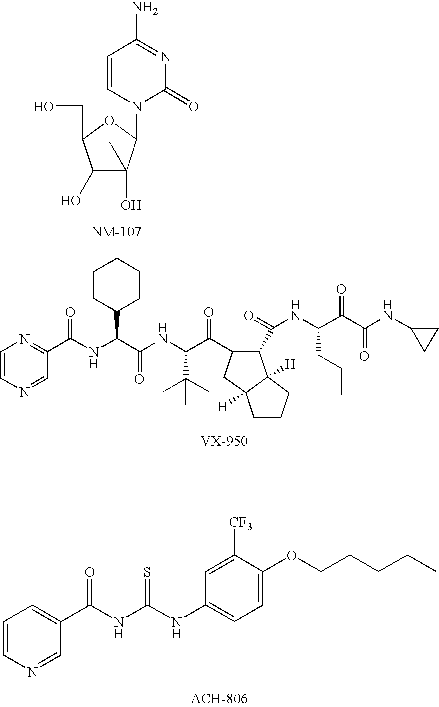

The structures of ACH-806, nucleoside NS5B polymerase inhibitor NM-107, and NS3 serine protease inhibitor VX-950 are shown above. These compounds are prepared using methods well known in the art of synthetic organic chemistry and fully characterized by ¹H-NMR, HPLC and LC-MS. All compounds are dissolved in DMSO and diluted in appropriate media at specified concentrations before addition to the test plates at the time of assay. IFN-alpha-2b (INTRON A, Schering, Kenilworth, N.J.) was purchased and was diluted in appropriate media at specified concentrations before addition to the test plates at the time of assay.

### Example 2

### Cell Lines

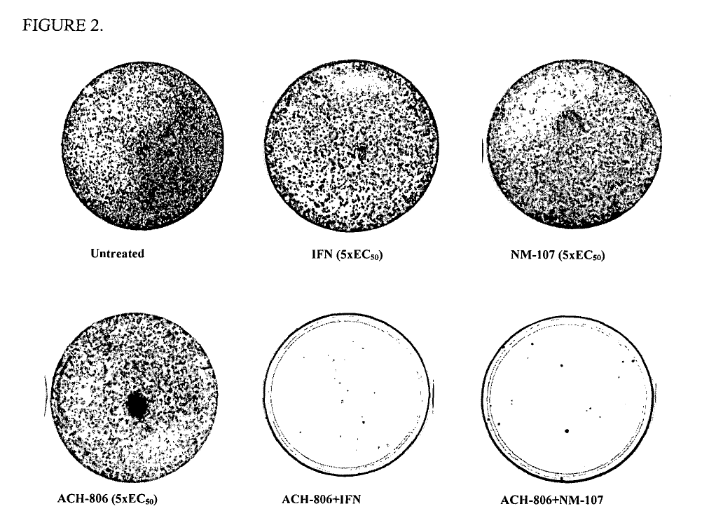

Huh-9-13 and Huh-luc/neo cell lines were obtained from Dr. Ralf Bartenschlager. These cell lines were established by transfection of replicon RNA molecules (subtype 1b, Con-1) as shown in FIG. 2. The cell lines were maintained in a complete medium [Dulbecco's modified minimal essential medium (DMEM) (Gibco BRL, Carlsbad, Calif.), 10% fetal bovine serum (FBS), and 1× nonessential amino acids (Gibco BRL, Carlsbad, Calif.)] with addition of 100 IU/ml penicillin, 100 μg/ml streptomycin and 0.5 mg/ml of G418 (Gibco BRL, Carlsbad, Calif.).

### Example 3

### Short Term Combination Study

Huh-luc/neo cells were seeded in 96-well plates at a density of 7000 cells/per well. One day after the cells were plated, the cells were treated with ACH-806, IFN-alpha-2b, VX-950 or NM-107 at various concentrations either alone or in combination. Dose-response curves for the individual compounds and their combinations were all run in quadruplicate. The concentrations of each drug span a similar range above and below EC50 so that equivalent antiviral activities are compared.

After 72 hours of incubation, the inhibition of HCV replicon replication in Huh-luc/neo cells was quantified by measurement of luciferase activity using a commercial luciferase assay (Britelite Ultra-High Sensitive Luminescence Reporter Gene Assay System, Perkin Elmer, Wellesley, Mass.) according to the manufacturer's instructions. Anti-HCV activity was expressed as the concentration that caused a reduction of luciferase activity (relative luminescence unit, RLU) by 50% (EC50) or 90% (EC90) in comparison to the untreated controls.

The toxicity of the compounds was evaluated by measurement of cell viability using a commercial CellTiter 96 Aqueous One Solution cell proliferation assay (Promega, Madison, Wis.). The cytotoxicity was expressed as the concentration that caused a reduction of OD490 value by 50% (CC50) in comparison to the untreated controls.

The experimental data were analyzed using CalcuSyn (Biosoft, Ferguson, Mo.), a computer program based on the method of Chou and Talalay (Chou, T. C. and P. Talalay, Quantitative analysis of dose-effect relationships: the combined effects of multiple drugs or enzyme inhibitors (1984) Adv. Enzyme Regul. 22: 27-55). In this model, the dose-effect curves for each drug or drug combination are converted to median-effect plots with the program. The effect of combination is then compared to that of a single agent. A combination index (CI) value for each experimental combination is calculated on the basis of the following equation:

CI=[(D)1/(Dx)1]+[(D)2/(Dx)2]+[(D)1(D)2/(Dx)1(Dx)2],

where (Dx)1 and (Dx)2 are the doses of drug 1 and drug 2 that have x effect when each drug is used alone, respectively, and (D)1 and (D)2 are the doses of drug 1 and drug 2 that have the same x effect when used in combination. By convention a CI of <0.9 is considered synergistic, a CI of ≧0.9 or ≦1.1 is considered additive, and a CI of >1.1 is deemed antagonistic.

The extent of synergy or antagonism was also assessed in a three-dimensional analytical method developed by Prichard and Shipman (Prichard, M. Shipman, K., A three-dimensional model to analyze drug-drug interactions, (1990) Antiviral Res. 14: 181-206). In this model, the theoretical additive effect is calculated from the dose-response curves of individual compounds by the equation Z=X+Y(1−X), where X and Y represent the inhibition produced by drug 1 alone and drug 2 alone, and respectively Z represents the effect produced by the combination of drug 1 and drug 2. The theoretical additive surface is subtracted from the actual experimental surface, resulting in a surface that would appear as a horizontal plane at 0% inhibition if the combination were merely additive. Any peak above this plane would indicate synergy, whereas any depression below it would indicate antagonism. The 95% confidence intervals for the experimental dose-response surface are used to evaluate the data statistically. The volume of the peak or depression is calculated to quantify the overall synergy or antagonism produced. Based on Prichard et al., volumes of synergy greater than 50 μM²% may be considered significant as may volumes of antagonism of less than −50 μM²%, wherein the units of the X and Y axis are both μM and the unit of the Z axis is the percentage of inhibition. Results

The combination consequences of ACH-806 with IFN, VX-950 and NM-107 were initially evaluated using a standard assay.

The inhibitory effect of ACH-806 in combination with one of IFN, VX-950 and NM-107 on replicon replication in Huh-luc/neo cells treated for 3 days was monitored by the luciferase activity, a reporter for the replicon copies. No cytotoxicity was observed for any of the compound treatments. The inhibitory effect on the replicon replication by these treatments was analyzed with both Chou and Talalay and Prichard and Shipman models. The results are summarized in Table 1. No antagonistic effect was observed. Furthermore, a synergistic antiviral activity of ACH-806 with VX-950 and NM-107 was observed.

TABLE 1
Combinatory antiviral activity after 3-days treatment

| Drug Combined | Volume (SD) μM² % | | Molar | CI (SD) at Replicon inhibition of | | | |
|---|---|---|---|---|---|---|---|
| with A CH-806 | Synergy | Antagonism | ratio | 50% | 75% | 90% | Overall |
| IFN | 4 (5) | 5 (3) | 1:10 | 1.12 (0.08) | 0.93 (0.15) | 0.81 (0.17) | Additive |
| | | | 1:20 | 1.04 (0.05) | 0.85 (0.09) | 0.73 (0.10) | |
| VX-950 | 119 (4) | 8 (3) | 1:10 | 0.86 (0.09) | 0.82 (0.11) | 0.83 (0.13) | Synergy |
| | | | 1:20 | 0.79 (0.06) | 0.61 (0.12) | 0.50 (0.15) | |
| NM-107 | 116 (77) | 2 (2) | 1:16 | 0.69 (0.02) | 0.72 (0.06) | 0.79 (0.12) | Synergy |
| | | | 1:32 | 0.67 (0.07) | 0.71 (0.05) | 0.83 (0.25) | |

### Example 4

### Long Term Combination Study

Huh-9-13 cells were seeded in 10 cm plates at a density of 10⁵ cells/plate and were treated with ACH-806 or NM-107 at the concentrations of approximately 5×EC50 values either alone or in combination for 9 days. In other repetitions of this experiment, treatment time varied from 7 to 10 days. Cells were passaged once every 3 days into new plates at the same density (10⁵ cells/plate) and with fresh compounds. No G418 was present during the 9 day treatment. On day 10, the cells were passaged again to new plates at a density of 10⁵ cells/plate, respectively, and were treated only with G418 for approximately 3 more weeks. During this time the colonies formed. During the 2˜3 weeks, the old media were replaced with fresh media twice a week. The colonies were fixed with 10% formaldehyde and stained with 2% crystal violet in 20% ethanol.

The long term combination consequences of ACH-806 with either IFN or NM-107 were also evaluated using a curing assay as described above. In this assay, the number of the cells in which the replicons still remained after the 9-day treatment was quantified based on the number of the colonies formed after G418 selection. The results are presented in FIG. 2. ACH-806 in combined with either IFN or NM-107 cured almost 99.99% of replicon-containing cells (4 log reduction) although each by itself was about 90% (1 log reduction).

What is claimed is:

1. A method of treating hepatitis C comprising providing a compound of Formula I or a pharmaceutically acceptable salt thereof, with at least one additional active agent to a patient infected with a hepatitis C virus, wherein Formula I is 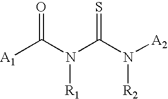
wherein
A₁ is an optionally substituted 3-pyridyl;
R₁ and R₂ are independently chosen from hydrogen and C₁-C₆alkyl; and
A₂ is phenyl substituted at the para position with C₃-C₈alkyl or C₃-C₈alkoxy and optionally substituted with one or more substituent independently chosen from halogen, hydroxy, cyano, C₁-C₆alkyl, C₁-C₆alkoxy, mono- and di-(C₁-C₆alkyl)amino, C₂-C₆alkanoyl, C₁-C₂haloalkyl, C₁-C₂haloalkoxy, and phenyl.

2. The method of claim 1 wherein the compound of Formula I is
(1-nicotinoyl-3-(4-(pentyloxy)-3-(trifluoromethyl)phenyl)thiourea);
(1-(4-(hexyloxy)phenyl)-3-nicotinoylthiourea);
(1-(5-bromonicotinoyl)-3-(4-(pentyloxy)-3-(trifluoromethyl)phenyl)thiourea);
(1-(6-(dimethylcarbamoyl)nicotinoyl)-3-(4-(pentyloxy)-3-(trifluoromethyl)phenyl)thiourea);
(1-(6-((dimethylamino)methyl)nicotinoyl)-3-(4-(pentyloxy)-3-(trifluoromethyl)phenyl)thiourea);
(1-(6-((4-methylpiperazin-1-yl)methyl)nicotinoyl)-3-(4-(pentyloxy)-3-(trifluoromethyl)phenyl)thiourea); or
(1-(4-(hexyloxy)phenyl)-3-(6-(morpholinomethyl)nicotinoyl)thiourea), or a salt of any of the foregoing.

3. The method of claim 1, wherein the compound of Formula I is (1-nicotinoyl-3-(4-(pentyloxy)-3-(trifluoromethyl)phenyl)thiourea).

4. A method of any one of claims 1 to 3, wherein the at least one additional active agent is a caspase inhibitor, a cyclophilin inhibitor, a cytochrome P450 monooxygenase inhibitor, a glucocorticoid, a hematopoietin, a fusion inhibitor, an entry inhibitor, a capsid inhibitor, a helicase inhibitor, a homeopathic therapeutic agent, an immunomodulatory compound, an immunosuppressant, an interleukins, an interferon enhancer, an IRES inhibitor, a monoclonal or polyclonal antibody, a nucleoside analogue, a non-nucleoside inhibitor, a P7 protein inhibitor, a polymerase inhibitor, a protease inhibitor, an RNA interference agent, a therapeutic vaccine, a TNF agonist, a tubulin inhibitor, an NS5A inhibitor, an NS4B inhibitor, a kinase inhibitors, a Toll like receptor agonist, and a sphingosine-1-phosphate receptor modulator.

5. A method of any one of claims 1 to 4 wherein the at least one additional active agent is an HCV protease inhibitor or HCV polymerase inhibitor.

6. A method of any one of claims 1 to 4 wherein the at least one additional active agent is VX-950 or valopicitabine.

7. A method of any one of claims 1 to 6 in which interferon is additionally provided to the patient.

8. A method of claim 1, wherein about 25 mg/day to about 2400 mg/day (1-nicotinoyl-3-(4-(pentyloxy)-3-(trifluoromethyl)phenyl)thiourea) and either about 200 mg/day to 400 mg/day valopicitabine or about 1200 mg/day to about 1800 mg/day VX-950 are provided to the patient.

9. A pharmaceutical combination comprising a compound or pharmaceutically acceptable salt of Formula I or a pharmaceutical acceptable salt thereof and at least one additional active agent, wherein Formula I is 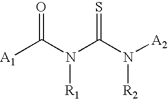
wherein
A₁ is an optionally substituted 3-pyridyl;
R₁ and R₂ are independently chosen from hydrogen and C₁-C₆alkyl; and
A₂ is phenyl substituted at the para position with C₃-C₈alkyl or C₃-C₈alkoxy and optionally substituted with one or more substituent independently chosen from halogen, hydroxy, cyano, C₁-C₆alkyl, C₁-C₆alkoxy, mono- and di-(C₁-C₆alkyl)amino, C₂-C₆alkanoyl, C₁-C₂haloalkyl, C₁-C₂haloalkoxy, and phenyl.

10. The pharmaceutical combination of claim 9, wherein the compound of Formula I is
(1-nicotinoyl-3-(4-(pentyloxy)-3-(trifluoromethyl)phenyl)thiourea);
(1-(4-(hexyloxy)phenyl)-3-nicotinoylthiourea);
(1-(5-bromonicotinoyl)-3-(4-(pentyloxy)-3-(trifluoromethyl)phenyl)thiourea);
(1-(6-(dimethylcarbamoyl)nicotinoyl)-3-(4-(pentyloxy)-3-(trifluoromethyl)phenyl)thiourea);
(1-(6-((dimethylamino)methyl)nicotinoyl)-3-(4-(pentyloxy)-3-(trifluoromethyl)phenyl)thiourea);
(1-(6-((4-methylpiperazin-1-yl)methyl)nicotinoyl)-3-(4-(pentyloxy)-3-(trifluoromethyl)phenyl)thiourea); or
(1-(4-(hexyloxy)phenyl)-3-(6-(morpholinomethyl)nicotinoyl)thiourea), or a salt of any of the foregoing.

11. The pharmaceutical combination of claim 9, wherein the compound of Formula I is (1-nicotinoyl-3-(4-(pentyloxy)-3-(trifluoromethyl)phenyl)thiourea) or a salt thereof

12. The pharmaceutical combination of any one of claims 9 to 11 wherein the at least one additional active agents is a caspase inhibitor, a cyclophilin inhibitor, a cytochrome P450 monooxygenase inhibitor, a glucocorticoid, a hematopoietin, a fusion inhibitor, an entry inhibitor, a capsid inhibitor, a helicase inhibitor, a homeopathic therapeutic agent, an immunomodulatory compound, an immunosuppressant, an interleukins, an interferon enhancer, an IRES inhibitor, a monoclonal or polyclonal antibody, a nucleoside analogue, a non-nucleoside inhibitor, a P7 protein inhibitor, a polymerase inhibitor, a protease inhibitor, an RNA interference agent, a therapeutic vaccine, a TNF agonist, a tubulin inhibitor, an NS5A inhibitor, an NS4B inhibitor, a kinase inhibitors, a Toll like receptor agonist, and a sphingosine-1-phosphate receptor modulator.

13. The pharmaceutical combination of any one of claims 9 to 12 wherein the at least one additional active agent is an HCV protease inhibitor or HCV polymerase inhibitor.

14. The pharmaceutical combination of claim 13, wherein the additional active agent is VX-950 or valopicitabine.

15. The pharmaceutical combination of any one of claims 9 to 14 wherein the combination is a unit dosage form.

16. A method of treating hepatitis C, comprising providing: (i) a pharmaceutical combination of any one of claims 9 to 15 in a container; and (ii) instructions for using the combination to treat a hepatitis C infection in a patient.
